(19) United States
(12) Patent Application Publication
(10) Pub. No.: US 2025/0325499 A1
(43) Pub. Date: Oct. 23, 2025

(54) IMMUNO-ABLATION TREATMENT FOR SOLID TUMORS

(71) Applicant: Northwestern University, Evanston, IL (US)

(72) Inventors: Dong Hyun Kim, Evanston, IL (US); Kijung Kwak, Evanston, IL (US)

(21) Appl. No.: 18/866,936

(22) PCT Filed: May 17, 2023
(86) PCT No.: PCT/US2023/067148

§ 371 Date: Nov. 18, 2024

Related U.S. Application Data
(60) Provisional application No. 63/343,497, filed on May 18, 2022.

(51) Int. Cl. A61K 31/12; A61K 31/045; A61P 35/00

(52) U.S. Cl. CPC A61K 31/12; A61K 31/045; A61P 35/00

(57) ABSTRACT

The present disclosure describes immune-ethanol ablation compositions comprising ethanol and curcumin for treatment of solid tumors. Also described herein are methods of using the immune-ethanol ablation compositions to treat solid tumor cancers and enhance anticancer immune activation.

### CROSS-REFERENCE TO RELATED APPLICATIONS

This application claims priority to, and the benefit of, U.S. Application No. 63/343,497 filed May 18, 2022, the content of which is incorporated herein by reference in its entirety.

### STATEMENT REGARDING FEDERALLY SPONSORED RESEARCH

This invention was made with government support from the National Institutes of Health under grant numbers R01CA218659 and R01EB026207. The government has certain rights in the invention.

### FIELD

The field of the invention relates to methods and compositions for treating solid tumors. More particularly the technology is directed to treating solid tumors with ethanol and curcumin.

### BACKGROUND

Cancer is the leading cause of death, and it causes major disease burden, worldwide. Specifically, solid tumors make up approximately 90% of adult cancers and can develop in almost all parts of the body including, but not limited to, breast, lung, prostate, colon, skin, bladder, kidney, liver and brain. Surgery is often the preferred first line of treatment for solid tumors, but for patients for whom surgery is not an option, ablation treatment is often used. Ablation techniques are less likely to cure the cancer than surgery but can still be helpful for some individuals.

Hepatocellular carcinoma (HCC) is the fourth-highest cause of cancer-related death globally.(1) Among the patients diagnosed with HCC, only 10%-20% are candidates for partial hepatectomy and liver transplantation.(2, 3) Over the years, local ablative methods have been developed for patients who are not surgical candidates because of multifocal disease, an inadequate liver remnant (size and function), or comorbid conditions. These techniques include percutaneous ethanol injection (ethanol ablation), percutaneous acetic acid injection, radiofrequency ablation (RFA), cryoablation, microwave ablation, laser-induced thermotherapy, high-intensity focused ultrasound (HIFU), and irreversible electroporation (IRE).(4) Among those ablation options, ethanol ablation employs direct infusion of ethanol into malignant tissue to induce necrosis through protein denaturation and cytoplasmic dehydration.(5) Due to a simple, safe, inexpensive, and highly-effective character of ethanol ablation technique with a low complication rate, ethanol ablation has also been successfully applied in the treatment of thyroid(6) and pancreatic tumors(7), cardiomyopathies(8), and adrenal metastases(9). Ethanol ablation is currently used to treat HCC with lesions up to 5 cm in diameter and yields 5-year survival rates comparable to surgical resection.(10, 11) Although ethanol-based tumor ablation is successful in treating HCC, the necessity of high-dose multiple treatment sessions, injection of large fluid volumes, and decreased efficacy in the treatment of non-capsulated tumors limit its applicability.(12) Moreover, the frequent recurrence of liver cancer post-ablation still impedes the final outcome.(13)

### SUMMARY

The disclosure provides an immune-ethanol ablation composition, and methods of their use, for treatment of solid tumors. In an aspect, the composition comprises ethanol and an anti-cancer immune modulator. In embodiments, the anti-cancer immune modulator is curcumin. In embodiments, the curcumin concentration is between about 0.1% w/v and about 5% w/v. In embodiments, the curcumin concentration in the composition is between about 0.1% w/v and about 1% w/v. In embodiments, the ethanol comprises between about 5% and about 30% of the composition. In embodiments, the ethanol comprises between about 5% and about 25% of the composition.

In another aspect, provided herein is a method for treating a solid tumor in a subject in need thereof, the method comprising injecting into the tumor any of the compositions comprising ethanol and curcumin described herein. In embodiments, the ethanol and the anti-cancer immune modulator are in an amount effective in combination to reduce the volume of the tumor. In embodiments, the treatment reduces the tumor volume by at least 50%. In embodiments, the tumor is a cancerous tumor. In embodiments, the tumor is a hepatocellular carcinoma (HCC) tumor, a thyroid tumor, a pancreatic tumor, or an adrenal tumor. In embodiments, the tumor is an HCC tumor. In embodiments, the subject is human. In embodiments, the ethanol and the anti-cancer immune modulator are in an amount effective in combination to prevent tumor formation or reduce the volume of tumors at other sites within the subject.

In another aspect, provided herein is a method for increasing an immune response to a tumor in a subject in need thereof, the method comprising injecting into the tumor any of the compositions comprising ethanol and curcumin described herein. In an embodiment, the ethanol and the anti-cancer immune modulator are in an amount effective in combination to increase the immune response to the tumor. In an embodiment, the immune response is characterized by decreased activation of at least one of STAT3, p65, and PD-L1. In an embodiment, the tumor is a cancerous tumor. In an embodiment, the tumor is a hepatocellular carcinoma (HCC) tumor, a thyroid tumor, a pancreatic tumor, or an adrenal tumor. In an embodiment, the subject is human.

### BRIEF DESCRIPTION OF THE DRAWINGS

FIGS. 1A-1C. Ethanol concentration and exposure time dependent cancer cell killing and viability. (A) Time and dosage-dependent cancer cell-killing effect of ethanol treatment. (B) PI and Annexin-V staining of N1S1 cells treated with various ethanol concentrations across different recovery times (0, 24, 48 h). (C) Quantification of live, early apoptosis, and late apoptosis (necrosis) of cells treated with various ethanol treatment conditions in each group. Data are shown as means±s.d.

FIG. 2. Western blot analysis of N1S1 hepatoma cells treated with various ethanol concentrations (1, 5, 15, 20, and 25%) at 4, 12, and 24 h post-treatment. The expression of iTME-associated proteins (p-STAT3, p-p65 and PD-L1) and ethanol treatment mediated GPX-4 expression were measured by western blot.

FIGS. 3A-3B. Integration of ethanol treatment with cellular delivery of curcumin anti-cancer immune modulator to treat cancer cells. (A) Cell viability of N1S1 cells treated with various ethanol-curcumin concentrations. The addition of curcumin effectively enhanced the cancer cell killing effect. (B) Western blot analysis of N1S1 hepatoma cells treated with various ethanol-curcumin concentrations (ethanol: 15 and 10% and curcumin: Cur1 (1% w/v), Cur2 (0.5% w/v), and Cur3 (0.1% w/v)) at 24 h post-treatment. The expression of iTME associated proteins (p-STAT3, p-p65 and PD-L1) and ethanol treatment mediated GPX-4 expression were measured by Western blot. Ethanol concentration was denoted as 15% and 10%. Curcumin concentrations were denoted as Curl (1% w/v), Cur2 (0.5% w/v), and Cur3 (0.1% w/v). Data are shown as means±s.d.

FIGS. 4A-4C. (A) Confocal images and (B) Flow cytometry assay of N1S1 cells treated with ethanol-curcumin solution. (C) Quantification of fluorescence intensity of curcumin by flow cytometry in each group treated with various concentrations of ethanol-curcumin solution and incubation time (2, 24, and 48 h) at post-treatment. Ethanol concentration was denoted as 15%, 10%, or 1%. Curcumin concentrations were denoted as Curl (1% w/v), Cur2 (0.5% w/v), and Cur3 (0.1% w/v).

FIGS. 5A-5E. Abscopal effect of a combination of ethanol and curcumin treated N1S1 tumor cells. (A) A schematic of timeline and tumor inoculation model to test the abscopal effect. (B) A table of details for experimental groups. (C) Typical MR images of N1S1 HCC grown with various groups at 8-day post-inoculation. (D) Measured tumor volume of right-side HCC tumors from each group (Control, Ethanol treated N1S1 cells, and Ethanol-Curcumin treated N1S1 cells). (E) Immune status of the right-side tumors by quantifying Tregs, CTLs, IL-17 T cells, and neutrophils in flow cytometry analysis. Data are shown as means±s.d. *P<0.05; **P<0.01.

FIG. 6. Protein expression levels of p-STAT3, p-p65, PD-L1, and GPX-4 in N1S1 cells treated with ethanol (25%-1%) quantified from Western blot across 4, 12, and 24 h time points. (n=3)

FIG. 7. In vitro flow cytometry analysis of PD-L1 expression on N1S1 cells treated with different concentrations of ethanol (0, 1, 5, and 10%).

FIG. 8. Schematic of intracellular curcumin to inhibit NF-κB and JAK-STAT pathway.

FIG. 9. Protein expression levels of p-STAT3, p-p65, PD-L1, and GPX-4 in N1S1 cells treated with a combination of ethanol and curcumin quantified from Western blot (n=3). Curl is 1% w/v, Cur2 is 0.5% w/v, and Cur3 is 0.1% w/v.

FIG. 10. Picture of N1S1 cells treated with curcumin (0.1% w/v) in various ethanol concentrations (40, 20, 10, 5, 2.5, and 1%).

FIG. 11. Confocal images on cell penetrating effect of ethanol (10%) on curcumin (0.5% w/v). Curcumin can be seen in the cells with co-treatment of ethanol. Control: no treatment.

FIG. 12. Representative gating strategies used for flow cytometry analysis of CD8⁺INF-γ⁺ T cells and Tregs (CD4⁺Foxp3⁺) in N1S1 tumors.

FIG. 13. Illustrative depiction of the general method of immuno-ethanol ablation with curcumin.

### DETAILED DESCRIPTION

Ethanol ablation is commonly used and well-accepted for the treatment of solid tumor cancers. As demonstrated in other ablation procedures, such as microwave ablation or cryoablation, ablation can induce necrosis and generate in situ tumoral antigens that may stimulate the immune system to inhibit potential recurrence during post-treatment immune surveillance. However, ablation is sometimes incomplete, and due to the tumor microenvironment, the immune response is not effective to fully reduce the tumor burden. The present disclosure describes a composition for an immune-ethanol ablation composition for treatment of solid tumors and methods for using the same that result in improved anticancer immune activation and treatment of solid tumors. The immune-ethanol ablation composition is more effective in reducing tumor volume and enhancing the immune response to tumors than traditional ethanol ablation treatment.

In an aspect, provided herein is a composition for use in ethanol ablation of tumors, the composition comprising ethanol and an anti-cancer immune modulator.

Ethanol refers to an alcohol with the formula C₂H₆O. Ethanol may also be called ethyl alcohol, alcohol, 64-17-5, C₂H₆O or CH₃CH₂OH. The composition may comprise 200 proof, 100 proof, among others. The ethanol may be used at a concentration in a range of about 1% to about 98%, about 96% or any concentration in between, including 96%, 95%, 90%, 85%, 80%, 75%, 70%, 65%, 60%, 55%, 50%, 45%, 40% 25%, 20%, 15%, 10%, 5%, 2.5% or 1%. In one example, the ablation composition comprises ethanol at about 5% to about 30%, preferably about 5% to about 25% of the composition.

Anti-cancer immune modulators for use in the compositions of the present invention include molecules that are capable of altering the immune cells within or that are recruited to the tumor microenvironment in order to produce an anti-tumor immune response. Suitable anti-cancer immune modulators are preferably able to modulate one or more immune mechanisms related to anti-tumor response including, but not limited to, an increase in CD8⁺IFN-γ⁺ cytotoxic T lymphocytes, increase in IL-17⁺CD4⁺ Th 17 cells, a decrease in Foxp3⁺CD4⁺ regulatory T cells, a decrease in neutrophils, an increase in one or more anticancer immune modulators (e.g., increased expression, activation, function of IFN-γ, IL-17, TNF-alpha, IL-1, IL-6, CXCL8, CCL2, CCL3, CCL4, CCL5, CCL11 or CXCL10), and/or decreased activation of JAK-STAT and NF-κβ pathways (e.g., decreased expression, activation, function, phosphorylation, or translocation to the nucleus of PD-L1, p65, STAT3, and STAT4, etc., and combinations thereof). The immune modulator may comprise one or more immune checkpoint inhibitors including but not limited to aPD-1, aPD-L1, CTLA-4, IDO and/or anti-VEGF inhibitors. The composition may comprise or consist of ethanol and an anti-cancer immune modulator in an amount effective in combination to provide an anti-cancer treatment.

In an exemplary embodiment, the anti-tumor immune modulator is curcumin. Curcumin ((1E,6E)-1,7-bis(4-hydroxy-3-methoxyphenyl)hepta-1,6-diene-3,5-dione) is also known as 458-37-7, diferuloylmethane, natural yellow 3, turmeric yellow, turmeric, Indian saffron or curcuma, and has the molecular formula C₂₁H₂₀O₆. The curcumin may be at least about 99.5% pure. Curcumin, a phytochemical, is a diarylheptanoid secondary plant metabolite isolated from turmeric and a member of the curcuminoid class which also includes demethoxycurcumin and bisdemthoxycurcumin. Curcumin is a potent anti-cancer immune modulator, and the examples demonstrate a synergistic anticancer effect with ethanol ablation to modulate the inflammatory tumor microenvironment (iTME).

The curcumin used herein may be a synthesized derivative or be modified to increase the bioavailability or solubility. Common derivates of curcumin are turmeric based and can include substitutions on the phenyl groups. For example, curcumin may be dissolved in ethanol, acetone, DMSO, a surfactant, co-surfactant or other organic solvent. Curcumin may also be dissolved in water, cell media, PBS or saline. Curcumin is dissolved by mixing in the solvent at room temperature. Dissolved curcumin may be used in a dose as suitable based on tumor volume and may include 0.1 ml, 0.15, 0.2 ml, 0.25 ml, 0.30 ml, 0.35 ml, 0.4 ml, 0.45 ml, 0.5 ml, 0.55 ml, 0.6 ml, 0.65 ml, 0.7 ml, 0.75 ml, 0.8 ml, 0.85 ml, 0.9 ml, 0.95 ml or 1 ml.

In the compositions described herein, the curcumin may be at least about 0.01% weight by volume (w/v) with the ethanol. The curcumin may be between about 0.01% w/v and about 5% w/v with ethanol, preferably between about 0.01% and about 2% w/v, or any concentration in between, e.g., 0.01%, 0.1%, 0.25%, 0.5%, 0.75%, 1%, 2%, 2.5%, 3%, 3.5%, 4%, 4.5% and 5%.

The composition may further comprise a pharmaceutically acceptable carrier. The term “pharmaceutically acceptable carrier” as used herein means a non-toxic, inert liquid filler, diluent, encapsulating material or formulation auxiliary of any type. The pharmaceutical compositions may be formulated for administration by, for example, injection. Therapeutic compositions typically are sterile and stable under the conditions of manufacture and storage. The pharmaceutically acceptable carrier used in the compositions described herein may be a diluent to dilute the ethanol to the proper percentage, such as sterile water, phosphate buffer saline, and the like. Additional suitable pharmaceutically acceptable carriers are known in the art, and include, but are not limited to, diluents, preservatives, solubilizers, emulsifiers, liposomes, nanoparticles and adjuvants, buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate or other carriers, according to the judgment of the formulator. Additionally, pharmaceutically acceptable carriers may be aqueous or non-aqueous solutions, suspensions, and emulsions. Examples of aqueous carriers include isotonic solutions, alcoholic/aqueous solutions, emulsions or suspensions, including saline and buffered media. Techniques, formulations, and pharmaceutically acceptable carriers may generally be found in Alphonso Gennaro, ed., Remington's Pharmaceutical Sciences, 18th Ed., (1990) Mack Publishing Co., Easton, Pa.

In a second aspect, provided herein is a method for treating a solid tumor in a subject in need thereof, the method comprising injecting into the tumor any of the compositions disclosed herein comprising ethanol and an anti-tumor immune modulator. The ethanol and the anti-cancer immune modulator may be in an amount effective in combination to reduce the volume of the tumor. In an embodiment, the method comprising injecting into the tumor any of the compositions disclosed herein consisting essentially of ethanol and an anti-tumor immune modulator. In an embodiment, the method consists essentially of injecting into the tumor any of the compositions disclosed herein comprising ethanol and an anti-tumor immune modulator.

The method of injecting the compositions described herein into the tumor may be referred to as “ablation therapy”. Ablation therapy is a type of minimally invasive procedure used to destroy abnormal tissue. Ablation therapy for tumors or cell masses, is performed percutaneously through a small hole in a probe that is targeted to the tumor. The ablation therapy may employ computed tomography (CT) imaging to pinpoint a tumor. Subsequently, a thin probe is inserted through the skin and maneuvered into the tumor. The ablation composition, typically ethanol, is then pumped through the probe into the tumor. As known in the art, injection is a way of administering a liquid using a needle and syringe. Ethanol ablation, simply injecting ethanol into lesions, is one of the most reliable ablation modalities. In the methods disclosed herein, the ablation composition comprises ethanol and an anti-cancer immune modulator. The compositions described herein may be administered to the tumor in ways other than injection. The compositions may also be administered via intra-arterial injection.

The method may reduce the volume of the tumor. Tumor volume is the size of a tumor or cell mass measured by the amount of space taken up by the tumor or cell mass. Tumor volume may be measured by an imaging device such as magnetic resonance imaging (MRI), Computed tomography (CT), X-ray or caliper or other means known in the art. The tumor volume or burden may be decreased at least 20%, preferably at least 50%, alternatively at least 25%, 30%, 35%, 40%, 45%, 50%, 55%, 60%, 65%, 70%, 75%, 80%, 85%, etc. The tumor volume may be decreased in an amount appreciable to increase the duration of survival of the subject.

As used herein, a “subject”, “patient”, or “individual” refers to an animal, which may be a human or non-human animal, in need of treatment. In particular embodiments, the subject is a human subject. A “subject in need of treatment” may include a subject having a disease, disorder, or condition that may be characterized as a tumor or cancer. As it is known in the art, a cancer is generally considered as uncontrolled cell growth. As used herein, the term “cancer” refers to any condition characterized by uncontrolled and or abnormal cell growth or uncontrolled division of abnormal cells in a part of the body. In some instances, the cancer may be characterized as a solid cancer and further by a tumor or lesion formation. Tumors are generally considered an abnormal mass, or growth in a part of a body caused by abnormal growth of tissue. A tumor may be benign or malignant, primary or metastatic.

As used herein, the terms “treating” and “to treat” mean to alleviate symptoms, eliminate the causation of resultant symptoms either on a temporary or permanent basis, and/or to prevent or slow the appearance or to reverse the progression or severity of resultant symptoms of the named disease or disorder, including reducing, preventing, ameliorating and/or improving the onset of the symptoms or complications, alleviating the symptoms or complications, or reducing or eliminating the disease, condition, or disorder. For example, treating cancer in a subject includes reducing, repressing, delaying or preventing cancer growth, reduction of tumor volume, and/or preventing, repressing, delaying or reducing metastasis of the tumor. Treating cancer in a subject also includes reducing the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer.

In a third aspect, provided herein is a method of increasing an immune response to a tumor in a subject in need thereof, the method comprising injecting into the tumor any of the compositions disclosed herein comprising ethanol and an anti-cancer immune modulator. The ethanol and the anti-cancer immune modulator may be in an amount effective in combination to increase the immune response to the tumor.

An “effective treatment” refers to treatment producing a beneficial effect, e.g., amelioration of at least one symptom of a tumor or a cancer. A beneficial effect can take the form of an improvement over baseline, i.e., an improvement over a measurement or observation made prior to initiation of therapy according to the method. A beneficial effect can also take the form of reducing, inhibiting or preventing further growth of a tumor or cancer cells; reducing, inhibiting or preventing metastasis or invasiveness of cancer cells; or reducing, alleviating, inhibiting or preventing one or more symptoms of a tumor, or cancer cells or metastasis thereof. Such effective treatment may, e.g., reduce patient pain, reduce the size of a tumor, reduce the size or number of cancer cells, reduce or prevent metastasis of cancer cells, or slow cancer cell growth. An “effective amount” refers to the amount or dose of the treatment, upon single or multiple dose administration to the subject, which provides the desired effect in the subject. Suitably the desired effect may be reducing the size or volume of a tumor or altering the tumor microenvironment in order to increase the anti-tumor immune response. An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the severity of the disease or disorder; the response of the individual subject; the particular treatment administered; the mode of administration; the bioavailability characteristics of the preparation administered; the dose regimen selected; the use of concomitant medication; and other relevant circumstances. The treatment methods disclosed herein may comprise injecting the composition into the tumor one or more times. The composition may be injected three or more times based on the tumor volume.

The tumor to be injected may be a cancerous tumor. Accordingly, the subject may be afflicted with a cancer, such as a sarcoma or a carcinoma. The cancer may be breast cancer, prostate cancer, colon cancer, squamous cell cancer, small-cell lung cancer, non-small cell lung cancer, ovarian cancer, cervical cancer, gastrointestinal cancer, pancreatic cancer, liver cancer, bladder cancer, hepatoma, colorectal cancer, uterine cervical cancer, endometrial carcinoma, salivary gland carcinoma, kidney cancer, vulval cancer, pancreatic cancer, thyroid cancer, hepatic carcinoma, melanoma, various types of head and neck cancer, Ewing sarcoma, etc. In exemplary embodiments, the tumor is hepatocellular carcinoma (HCC), a thyroid tumor, a pancreatic tumor, a renal tumor, a prostate tumor, a lung tumor, a colorectal tumor, a colon tumor, or an adrenal tumor or metastasis.

In a fourth aspect, provided herein is a method of modulating the inflammatory tumor microenvironment (iTME) in a subject in need thereof, the method comprising injecting into the tumor any of the compositions disclosed herein comprising ethanol and an anti-cancer immune modulator.

As used herein, “the tumor microenvironment” is the environment around a tumor, including the surrounding blood vessels, immune cells, fibroblasts, signaling molecules, antibodies, and the extracellular matrix (ECM). Immune cells in the tumor microenvironment include myeloid derived suppressor cells and tumor associated macrophages, neutrophils, tumor infiltrating lymphocytes and T cells. The tumor microenvironment is an immunosuppressive environment that limits anti-tumor immune response. Thus, the compositions and methods of the present disclosure, may alter the tumor microenvironment from an immunosuppressive environment to an immunologically active environment that allows for an increased anti-tumor immune response.

The methods may cause alterations in the microenvironment that lead to an increase in the anti-cancer or anti-tumor immune response. The anti-cancer or anti-tumor immune response can include an increase in CD8⁺IFNγ⁺ cytotoxic T lymphocytes and IL-17⁺CD4⁺ Th 17 cells. In addition, the anti-tumor immune response can include a decrease in Foxp3⁺CD4⁺ regulatory T cells and neutrophils compared to non-treated subjects or subjects treated with ethanol ablation alone. Anticancer immune activation may be further characterized by increased expression, activation, or function of IFN-γ, IL-17, TNF-a, IL-1, IL-6, CXCL8, CCL2, CCL3, CCL4, CCL5, CCL11 or CXCL10. Anticancer immune activation may also be characterized by decreased activation of JAK-STAT and NF-κ62 pathways including decreased expression, activation, function or phosphorylation or translocation to the nucleus of CCL22, PD-L1, p65, STAT3, and STAT4 can be the anti-tumor immune response.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patents, and patent applications mentioned in this specification are herein incorporated by reference to the same extent as if each individual publication, patent, and patent application was specifically and individually indicated to be incorporated by reference.

### EXAMPLES

### Integrating Ethanol Infusion with Cellular Delivery of Immune Modulator for Local Ablation of Hepatocellular Carcinoma

Tumor ablation procedures can generate in situ tumoral antigens, which may aid in inhibiting potential recurrence in post-treatment surveillance by enrolling the immune system.(14) However, finding a practical and efficient strategy to boost the anticancer immune response is still a challenge. In particular, ablation therapy mediated inflammatory tumor microenvironment (iTME) plays a vital factor in oncogenesis and cancer progression.(15, 16) Even though the mechanisms of iTME-related oncogenesis and cancer progression still remain elusive, there is a growing consensus that the abnormal iTME formed after the ablation procedure enhances proliferation of cancer by causing immune suppression.(17) Thus, the modulation of iTME could improve anticancer immune response in the ablation therapies. Currently, delivery of immunotherapy-related agents, such as immune checkpoint blockade, small molecular inhibitors of the immunosuppressive activating enzyme, and vaccines for promoting immunostimulatory effects, is a widely used method for modulating iTME. However, it is still a challenge to efficiently deliver the agents to the targeted lesion and achieve a coherent therapeutic procedure. Thus, additional efforts are needed to establish a sophisticated strategy that rationally integrates therapy procedures with the delivery of immunomodulatory adjuvants.

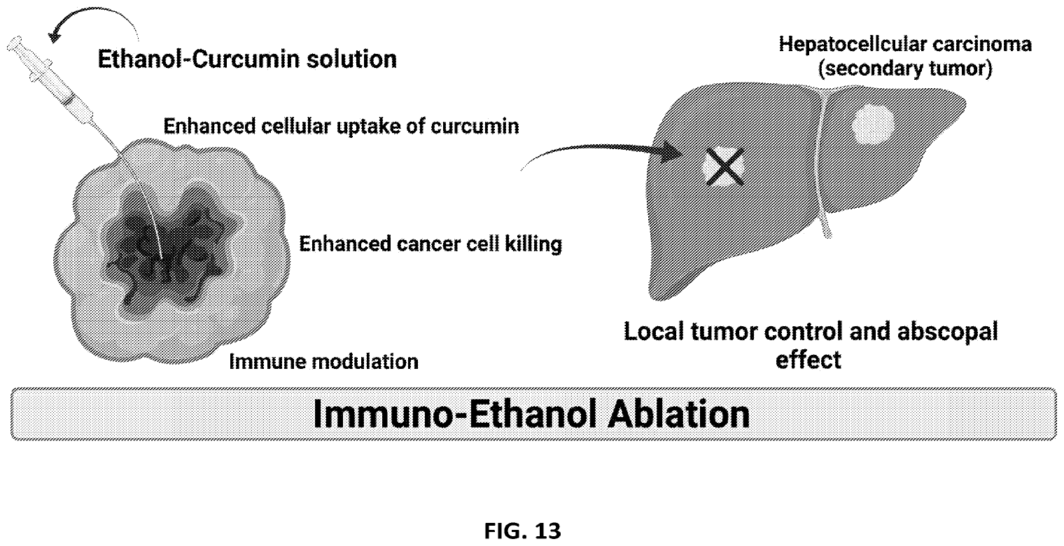

Herein, ethanol ablation combined with infusion of an immune modulation agent was proven to achieve a synergistic anticancer treatment and iTME modulation in a rat liver cancer model (FIG. 13). There are three impacts demonstrated. First, synergistic effect was achieved by combining ethanol ablation with curcumin, which is a potent anti-cancer immune modulator of iTME. Second, the ethanol ablation procedure contributed to enhanced cellular uptake of curcumin, which opens up a new horizon to integrate therapy procedures with the delivery of immune modulators for follow-up anticancer immune response. Third, ethanol-assisted local cellular delivery of curcumin modulated ablation-induced iTME, which is the main barrier to improving the survival rate. These findings demonstrate the potential of ethanol ablation-based combination immunotherapy and underscore the importance of the coherent therapeutic procedure in the outcome of ablation-based anticancer treatment.

### Materials and Methods

### Materials

Curcumin and ethanol (200 proof, ≥99.5%) were purchased from Sigma-Aldrich (St. Louis, MO) and used without additional purification. Curcumin-infused ethanol formulation was prepared by simple mixing of curcumin powder in ethanol until complete dissolution.

### Cell Viability Assay

N1S1 cells (1×10⁴/mL) were seeded onto 96-well plates. After 18-24 h incubation, the cells were treated with ethanol or curcumin-ethanol solution for 5 min or 10 min to assess viability and recovery response. Then the cells were washed with PBS and replenished with fresh media. After 24 h or 48 hr incubation, the cells viability was measured with CCK-8 assay.

### Western Blot Analysis

N1S1 cells were seeded in the 8 cm culture dishes and cultured at 5% CO2, 37° C. overnight. The cells were treated with various groups for 10 minutes: ethanol (25, 20, 15, 10, and 5%) for 4, 12, and 24 h, and 15% ethanol+Cur1, 10% ethanol+Cur1, 15% ethanol+Cur2, 10% ethanol+Cur2, 15% ethanol+Cur3, and 10% ethanol+Cur3 for 24 h. Curcumin concentrations were denoted as Cur1 (1% w/v), Cur2 (0.5% w/v), and Cur3 (0.1% w/v). No treatment of N1S1 was used as control. The cell lysates were collected and analyzed by Western blotting according to the manufacturer's instructions. Antibodies were used listed as below: GPX4 Rabbit anti-Human, Mouse, Rat, Polyclonal, Invitrogen™, catalog number: PIPA579321, dilution 0.1-0.5 μg/mL; p-STAT3 Rabbit mAb, cell signaling, catalog number: 9145, dilution 1:2000; p-p65 Rabbit mAb, cell signaling, catalog number: 3033, dilution 1:1000; PD-L1 Polyclonal Antibody, Invitrogen™, catalog number: PA5-20343, dilution 0.5-2 μg/mL; β-Actin (8H10D10) Mouse mAb (HRP Conjugate), cell signaling, catalog number: 12262, dilution 1:1000.

### Confocal Images

Briefly, the N1S1 cells (1×10⁵/mL) were cultured in a confocal dish (MatTek, 35 mm dish with 14 mm glass bottom). The cellular nucleus was stained with DAPI. Cell membranes were stained with Dil red (D282) following the protocol from ThermoFisher. After rinsing with PBS, the cells were treated with curcumin-ethanol solution. The cells were then fixed by 4% paraformaldehyde and visualized with a confocal microscope (Nikon AIRS). Curcumin was excited with a wavelength of ˜458 nm (emission wavelength ˜530 nm).

### Flow Cytometric Analysis of Annexin-V/PI and Curcumin Uptake

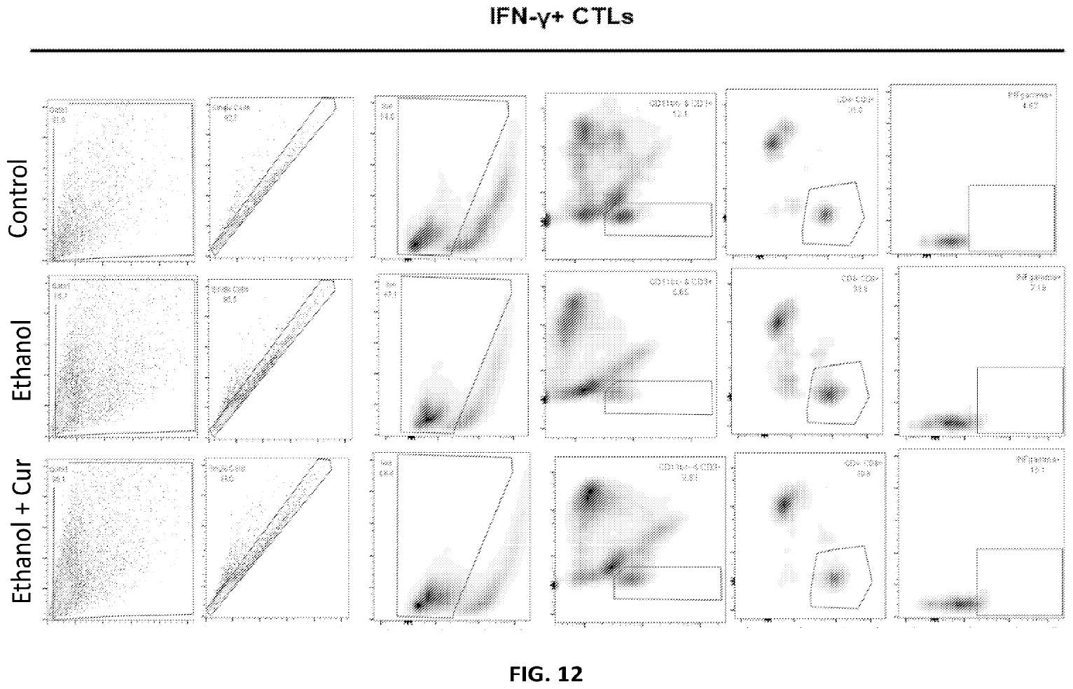

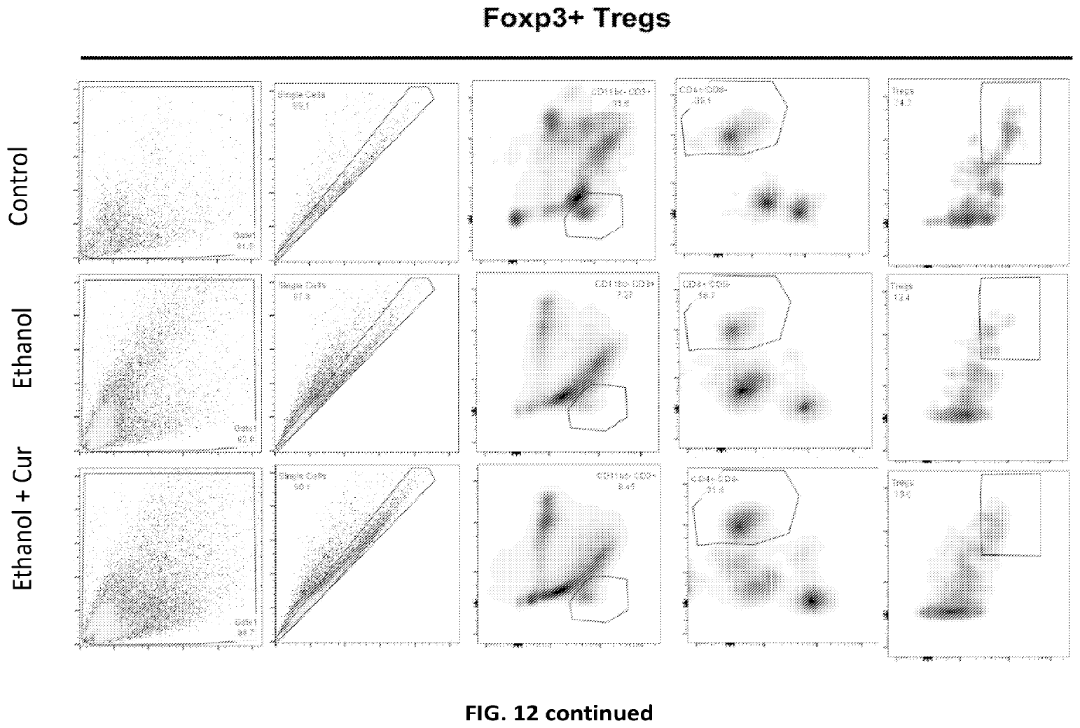

N1S1 cells were seeded onto the six-well plate (2×10⁵/well). At different time points (0, 24, and 48 h) post co-incubation with ethanol, the cells were washed with cold PBS and stained with FITC-annexin-V and propidium iodide (PI) according to the manufacturer's (ThermoFisher) instructions. The fluorescence of curcumin uptake was measured after the treatment of cells by curcumin-ethanol solution as described previously. After washing the cells with PBS, they were fixed with 4% formalin at room temperature for 15 minutes. The results were examined by flow cytometry. Representative gating strategies used for flow cytometry analysis of CD8+INF-γ+ T cells and Tregs (CD4+Foxp3+) in N1S1 tumors are shown in FIG. 12.

### In Vivo Evaluation of Abscopal Effect of Ethanol-Curcumin Treatment in Rats

The study protocol was reviewed and approved by the Institutional Animal Care and Use Committee at Northwestern University. N1S1 rats were randomly divided into three treatment groups (4-5 rats per group). The abscopal effect was tested by implanting N1S1 cells with/without treatment into the right and left liver lobes using mini-laparotomy procedures(18, 19). To evaluate the potential abscopal effect of ethanol-curcumin treatment, the right lobe of rat liver (Right-side) and left lobe of rat liver (Left-side) were simultaneously inoculated N1S1 (5 million) and a mixture of treated N1S1 (2.5 million, 10% ethanol only or 10% ethanol plus Cur2) and N1S1 (2.5 million), respectively. The cells were washed with PBS three times prior to inoculation. The rats in the control group were only inoculated in the right lobe of the liver with 5 million cells. The right-side tumors that were established for testing abscopal effect were collected for the immune status assay while the only tumor in the control group tested as the baseline. The tumor size was measured with 7.0-T ClinScan high-field small animal MR imaging system with a commercial rat coil (Bruker Biospin, Billerica, Massachusetts). Tumor volume was calculated as volume=(tumor length)×(tumor width)²/2.

### Investigation of In Vivo Anti-Cancer Immune Activation of Ethanol-Curcumin Treated Cells

To test in vivo tumor neutrophil cell populations, rats were sacrificed, and tumors were harvested at 8 days post treatment and examined by flow cytometry after immunofluorescence staining with anti-CD45-BV605, anti-Granulocytes-PE, and anti-CD11b/c-PerCP/eF710. To determine the intra-tumoral infiltration of CD8+ (CD3+CD4-CD8+), lymphocytes were stained with anti-CD45-BV605, anti-CD3-FITC, anti-CD11b/c-PerCP/eF710, anti-CD4-APC/Cy7, anti-CD8-V450, and anti-IFN-gamma-AF647. To determine the intra-tumoral infiltration of CD4+ (Treg), lymphocytes were stained with anti-CD45-BV605, anti-CD3-FITC, anti-CD11b/c-PerCP/eF710, anti-CD4-APC/Cy7, anti-CD8-V450, anti-CD25-PE, and anti-Foxp3-APC. All antibodies were diluted with the ratio of 1:100 and signal color beads (OneComp eBeads™ Compensation Beads) and unstained bead were used for compensation.

### Data Analysis

Data were presented as mean±standard deviation (s.d.) and analyzed in R Studio (statistical software). Student's t-test was used to assess differences between means. P<0.05 was considered statistically significant.

### Results and Discussion

### Cellular Response to Ethanol Treatment

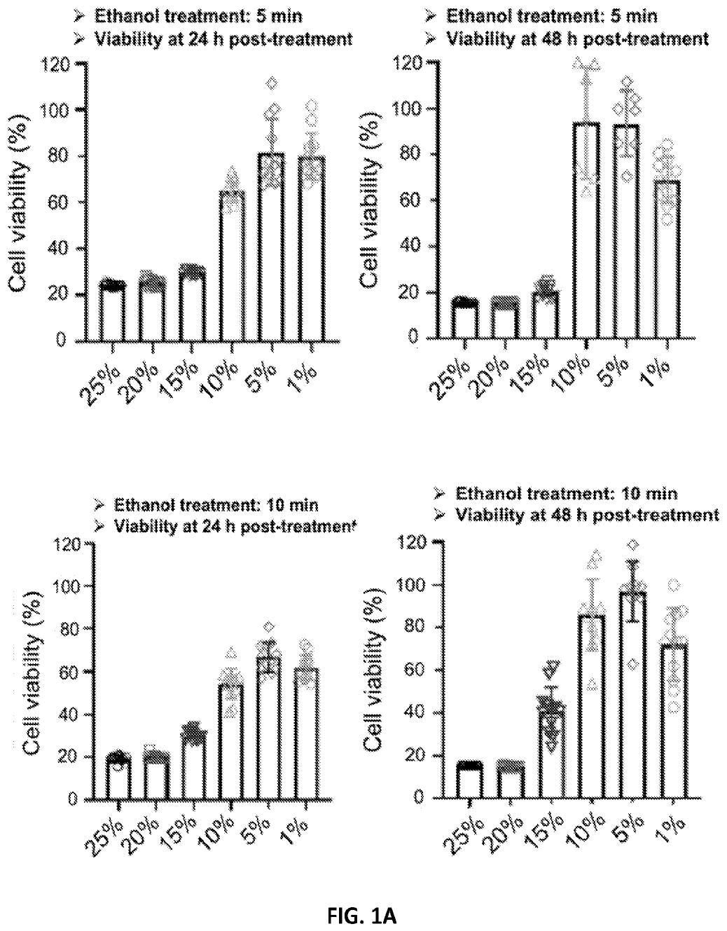

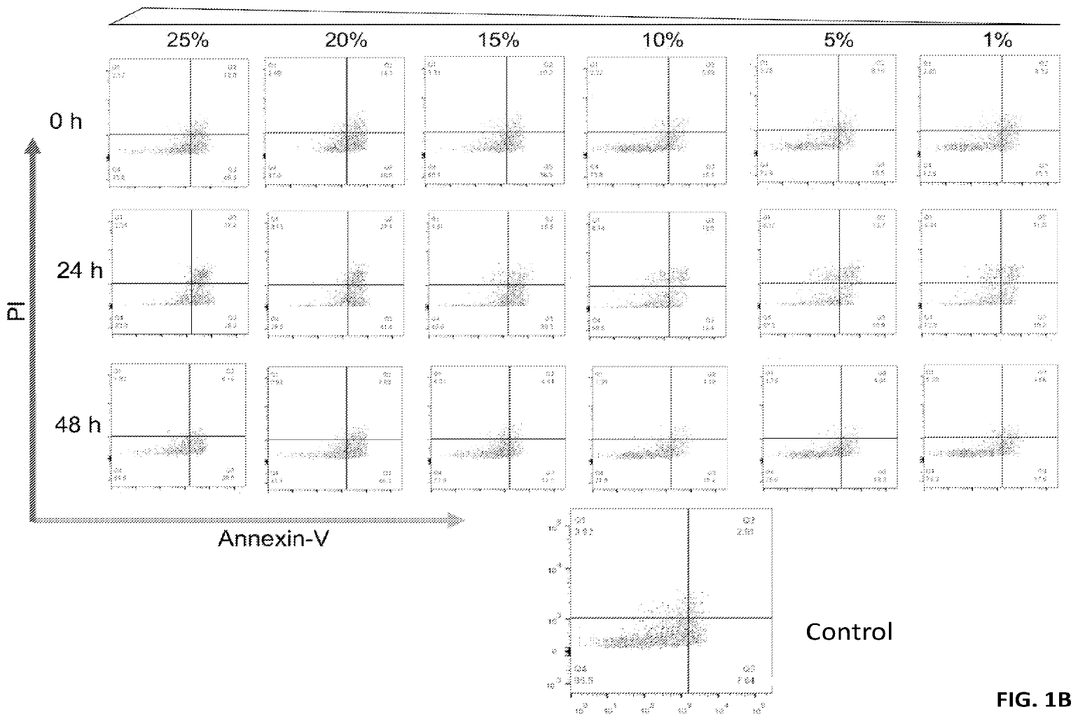

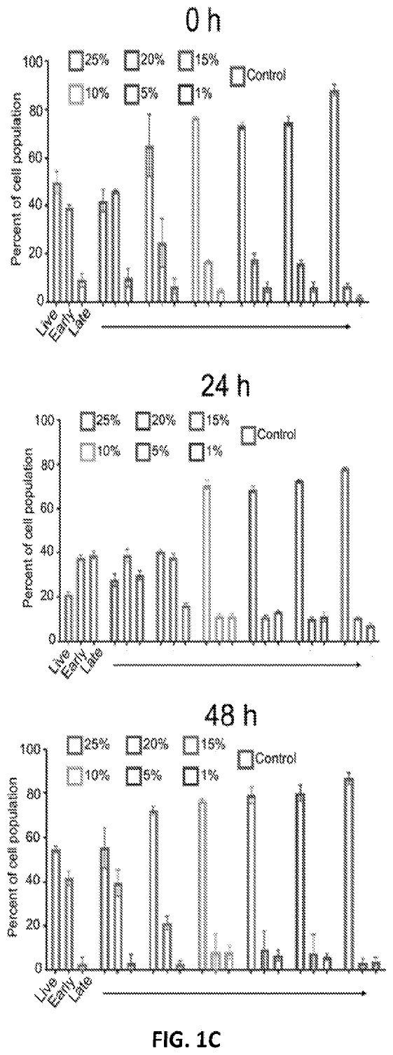

Cancer cell death and viability after ethanol treatment is dependent on the concentration of ethanol and the exposure time. To determine the influence of concentration and coincubation time of ethanol on the anticancer effect of the ethanol ablation procedure, CCK-8 kit was used to analyze N1S1 cell viability at 24 h and 48 h post-treatment by ethanol treatment with various concentrations and coincubation periods. As shown in FIG. 1A, dosage-dependent inhibition of ethanol against N1S1 hepatoma cells was confirmed in both 5-min and 10-min ethanol coincubation groups. The relative high volume-concentration of ethanol at 25% and 20% led to efficient growth inhibition in both groups, while treatment with the lower-concentration range of 15% to 1% resulted in a modest cell-killing effect. The recovery of cell viability at 48 h post-treatment in both 5-min and 10-min ethanol coincubation groups further demonstrated the inefficient cell killing of ethanol at lower concentrations. As compared to the value tested at 24 h, it was also found that the error range at 48 h post treatment was extended in the group with the same coincubation time (5 min or 10 min), indicating variance in proliferation as cells resume recovery. This indicates that current percutaneous ethanol injection inducing ethanol concentration gradient from tumoral injection site to peripheral tumor region results in incomplete cancer cell killing, leading to tumor recurrence post-ethanol ablation.(20)

Next, the annexin V-FITC/PI assay was utilized to further investigate the cell-killing effect of ethanol against N1S1 (FIGS. 1B and 1C). According to CCK-8 test, the longer coincubation period, 10 min, contributed to a more efficient cell-killing efficiency; therefore, the 10-min coincubation period was used to perform the following analysis. Dosage-dependent increase in both early apoptosis population (Annexin-V positive and PI negative) and late apoptosis population (Annexin-V positive and PI-positive) was observed. This effect was even found at 0 h post coincubation, which indicated the immediate cell-killing effect of ethanol. At relatively high ethanol concentrations (25% and 20%), direct protein denaturation of dead cell structure by the ethanol treatment likely led to a decreased late apoptosis population at 48 h compared to the populations analyzed at 0 h and 24 h post-treatment. The live cell populations (Annexin-V negative and PI negative) in groups treated by relatively high ethanol concentrations (25% and 20%) remained low through all time points. In contrast, higher live cell populations were found at 48 h in the groups treated with lower ethanol concentrations (15%, 10%, 5%, 1%), which is in line with the findings in FIG. 1A.

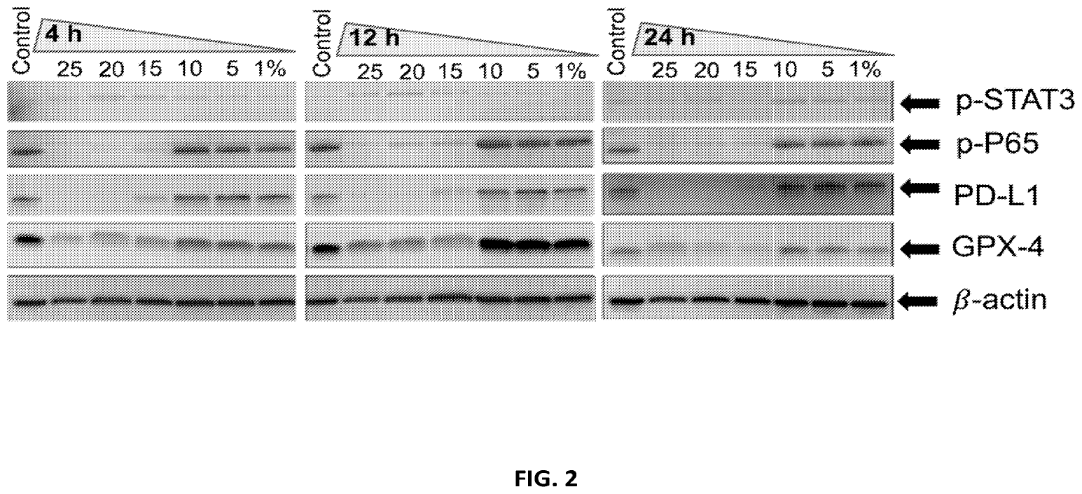

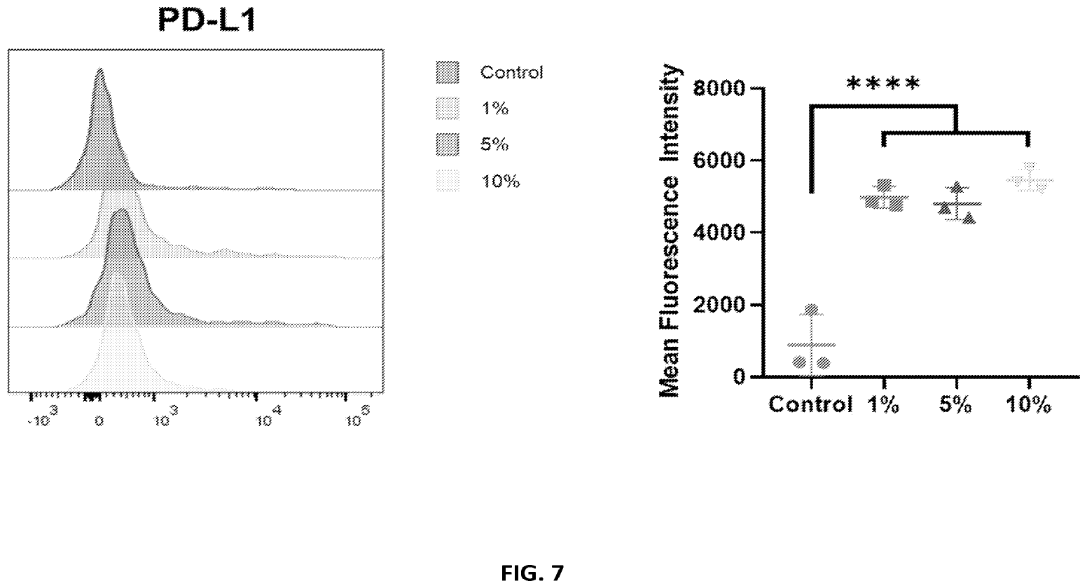

Western blot assay was then used to identify specific molecules associated with cancer cell response to the ethanol treatments. Ethanol treatment can enhance the production of oxygen-reactive species and decrease the level of endogenous antioxidants, leading to an increase in lipid peroxidation.(21) Herein, the level of GPX-4, which is a critical component of a concerted anti-peroxidant mechanism, was observed by western blot assay. Relatively high ethanol concentrations (25, 20, and 15%) fixed the cells and resulted in consistently low GPX-4 levels in all 4 h, 12 h, and 24 h time points. However, relatively low ethanol concentrations (10, 5, and 1%) increased the GPX-4 level at 12 h post-treatment compared to 4 h post-treatment, indicating a defensive response to the oxidative stress caused by ethanol. Then, at 24 h post-treatment, the expression of GPX-4 was reduced to normal levels, indicating the recovery of treated cells after treatment with the low ethanol concentrations. This is in agreement with the results of the cell viability and Annexin-V/PI assays (FIG. 1). Furthermore, ethanol treatment (10, 5, and 1%) resulted in consistent activation of p-STAT3 and p-p65, and upregulation of PD-L1 in N1S1 hepatoma cells, contributing to the immune suppressive tumor microenvironment after the incomplete ethanol ablation (FIG. 2 and FIG. 7).(22-24)

Taken together, these results indicate that since small ethanol molecules can easily diffuse out of the tumor tissues, the ethanol concentration and exposure time to cancer cells can be varied.

Both the cell killing effect and the potential cell survival in the ethanol ablation procedure are ethanol concentration-dependent and exposure time-dependent. Importantly, incomplete cell death in the relatively low ethanol concentration treatment promotes iTME, which can be pro-oncogenic for the tumor recurrence and tumor progression.(25-28). The modulation of iTME induced by ethanol ablation is therefore essential to enhance the therapeutic outcome of ethanol ablation and further induce synergistic anti-cancer immune response.

### Anti-Cancer Effect and iTME Modulation of Ethanol-Curcumin Treatment

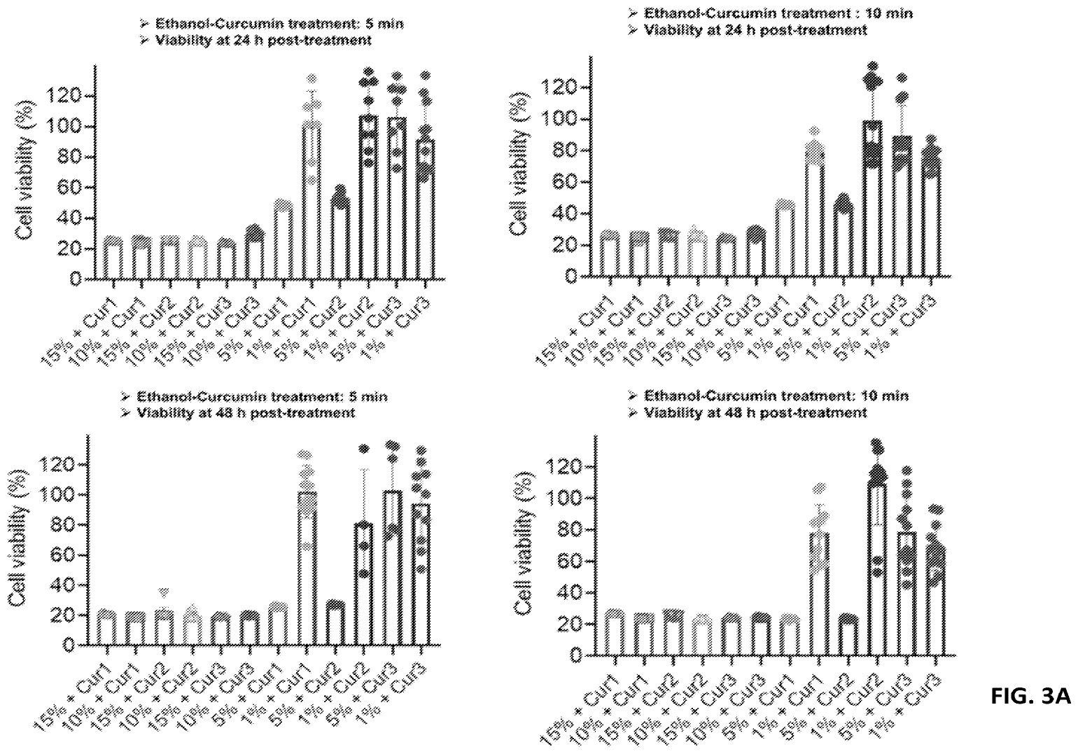

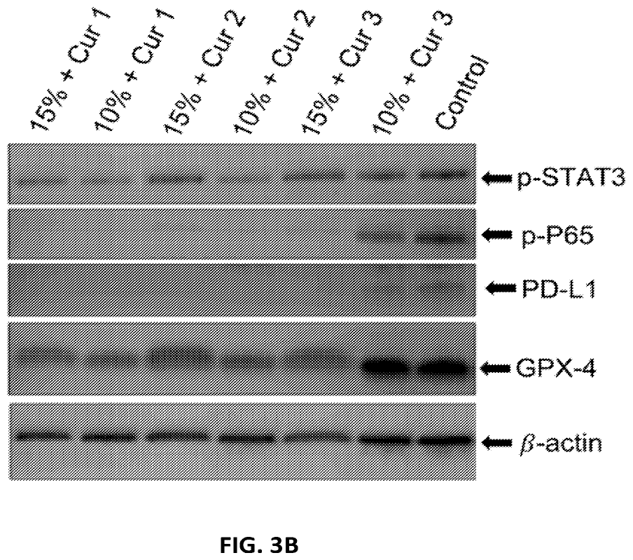

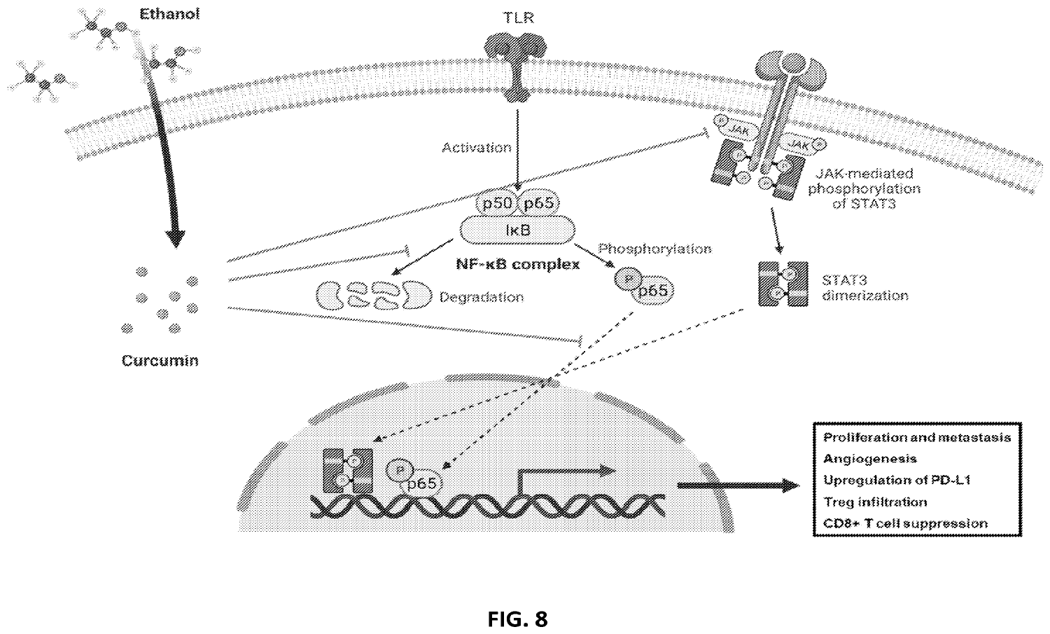

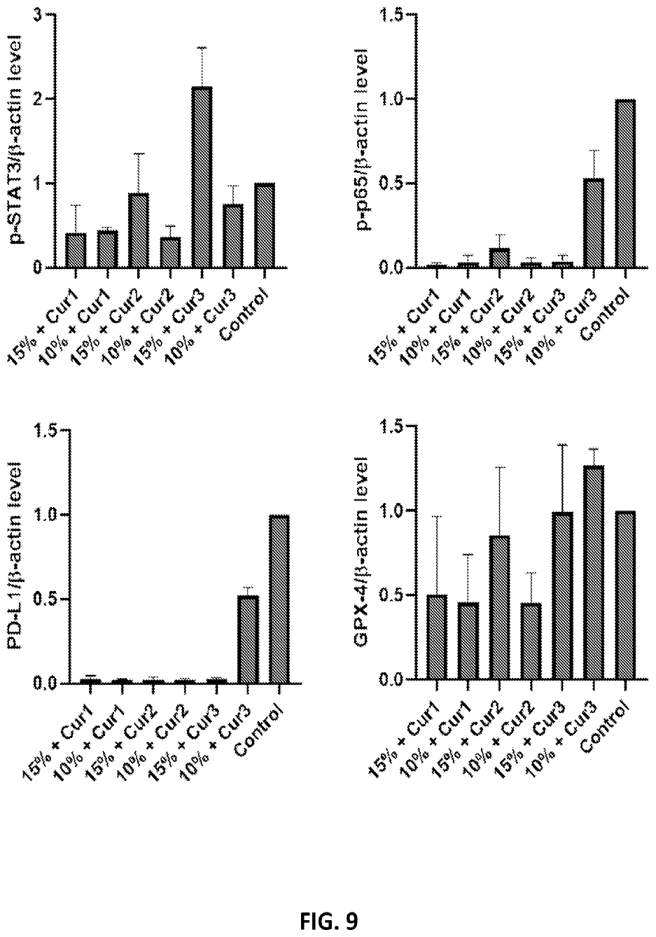

To address the potential incomplete ablation and iTME resulting from ethanol treatment alone, an anticancer molecule and immune modulator, curcumin, was integrated in the ethanol ablation procedure to treat N1S1 cancer cells. The inventors first verified the cell-killing effect of adding curcumin to ethanol. As shown in FIG. 3A, cell viability significantly decreased under the co-treatment of curcumin and ethanol with ethanol concentrations (15% and 10%) compared to ethanol treatment alone (FIG. 1A). The enhanced cell-killing effect could be achieved with a combination of ethanol and a high concentration of curcumin, even as ethanol concentration was reduced to 5% and 1%. These results demonstrate augmentation of the anticancer effect of combined ethanol with curcumin, as well as the potential to control the toxicity of ethanol. Moreover, the combination of ethanol and curcumin demonstrate the modulation of p-STAT3, p-p65, and PD-L1, which can be further utilized to change iTME induced by ethanol treatment alone (FIGS. 2, 3B and 9). Curcumin is a known inhibitor of the JAK-STAT pathway and the NF-κB pathway(29, 30), and as illustrated in FIG. 8, it blocks the translocation of p65 and STAT3 to the nucleus, resulting in iTME modulation.

### Enhanced Cellular Uptake of Curcumin in Ethanol-Curcumin Treatment

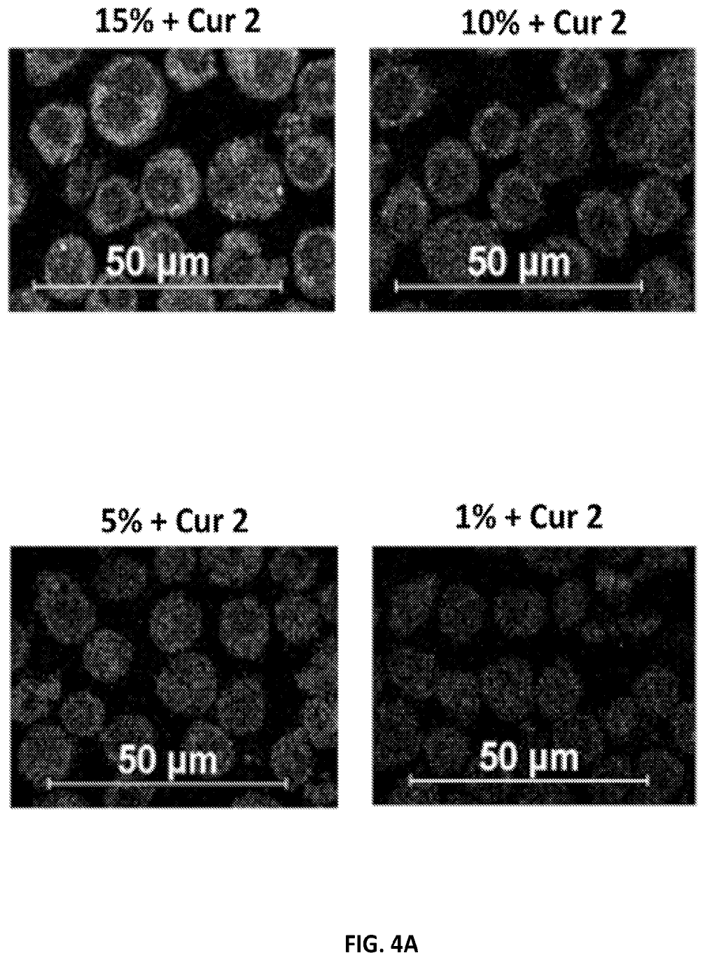

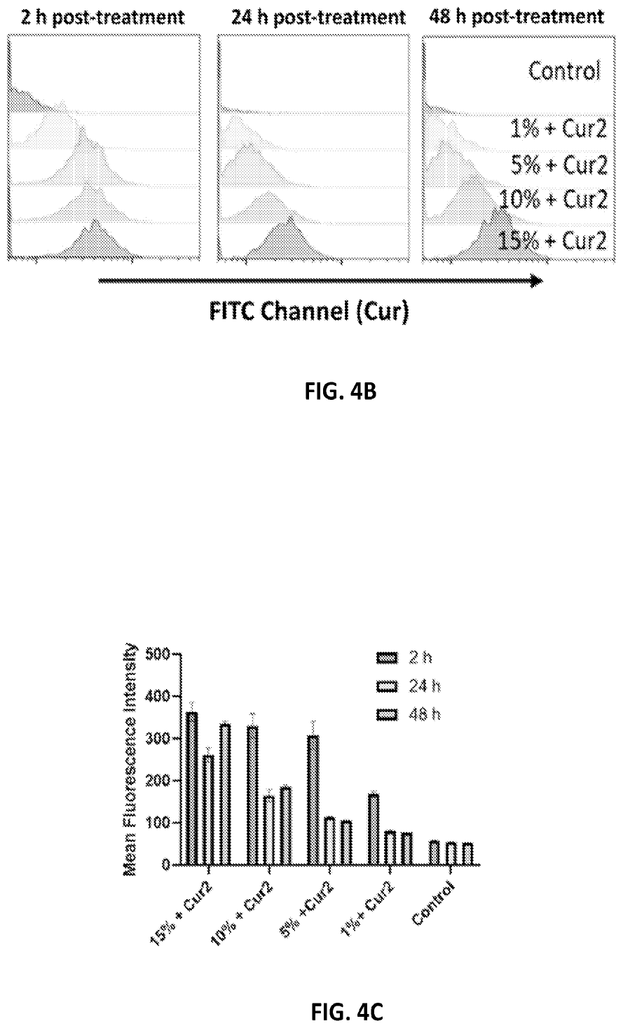

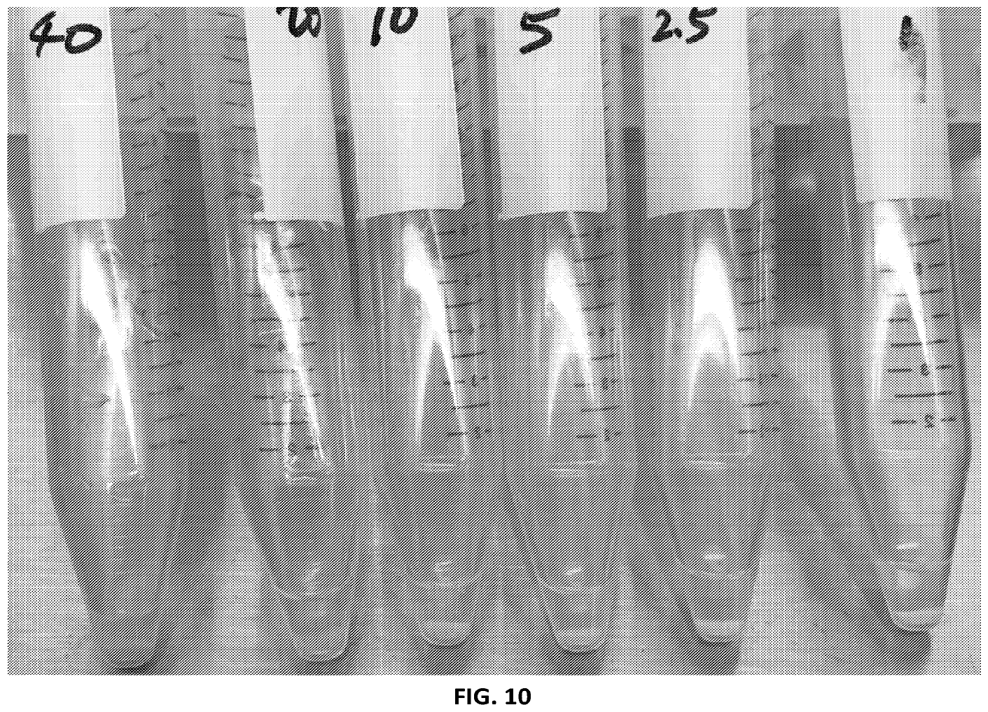

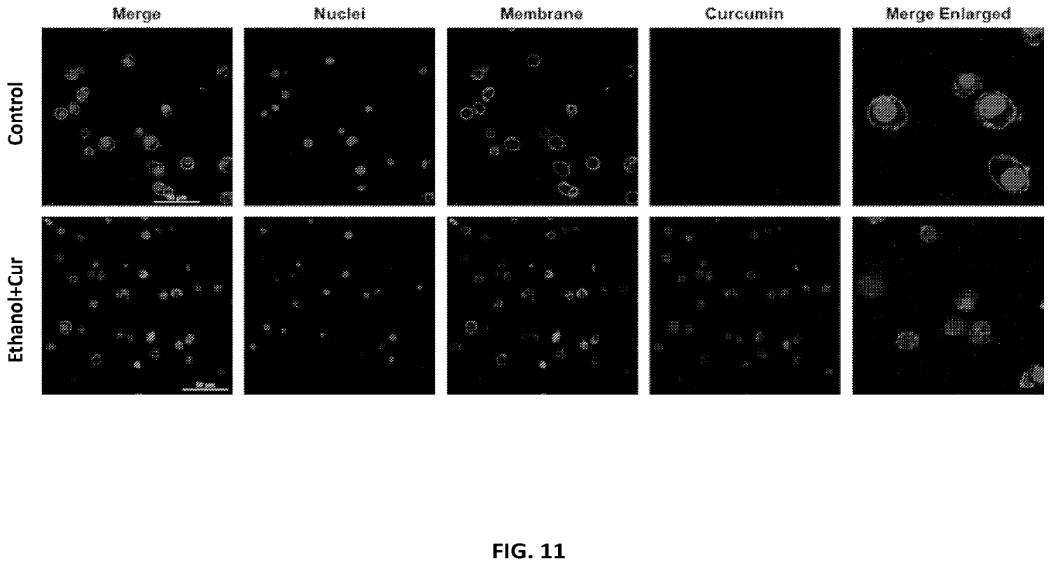

In addition, it was found that the ethanol ablation enhanced cellular delivery of curcumin. A dosage-dependent cellular delivery of curcumin during ethanol ablation was shown in FIG. 4A and FIG. 10. A higher concentration of ethanol resulted in brighter green fluorescence signal, which indicated a higher cellular uptake of curcumin. This may be due to the penetration effect of ethanol over cell membranes (FIG. 11). Flow cytometry was used to quantify the change of curcumin fluorescence. As shown in FIGS. 4B and 4C, the high ethanol concentration (15%) led to the stronger green fluorescence that persisted for a long period of 48 h, which demonstrates pause of cellular life. This result is consistent with the CCK-8 assay shown in FIG. 3A. Lowering the concentration of ethanol (e.g., 10%, 5%, and 1% ethanol) results in a decrease in fluorescence at 48 h, which demonstrates the continuous metabolism of curcumin by the cells. The metabolism of curcumin indicates the potential iTME modulation by the adjuvant curcumin.

### Abscopal Effect and Anti-Cancer Immune Response of Curcumin-Ethanol Ablation

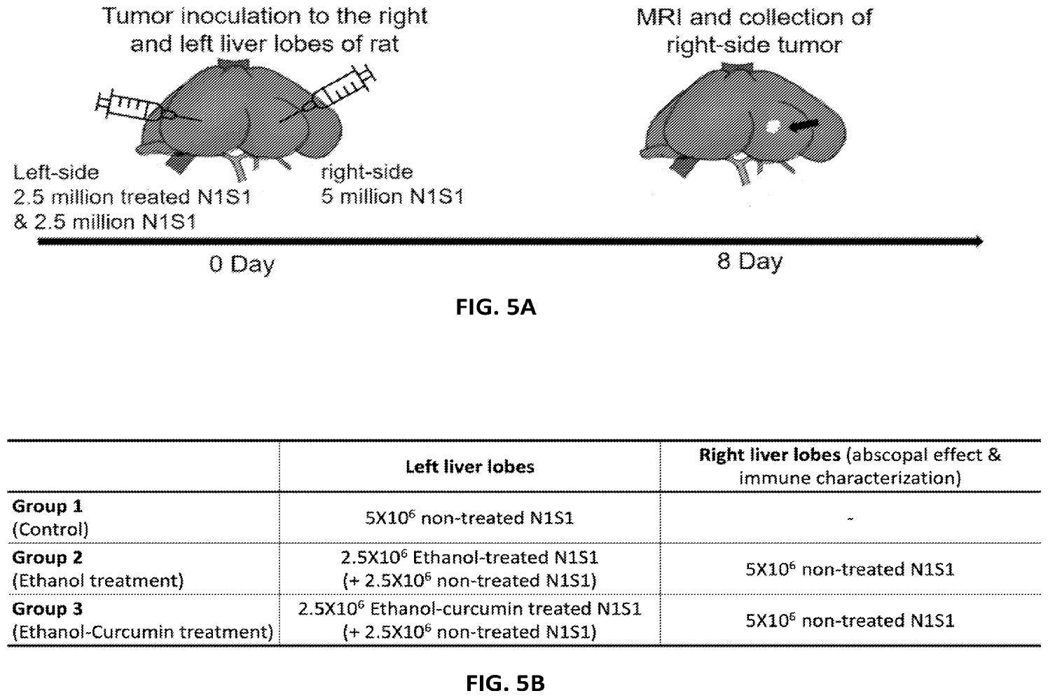

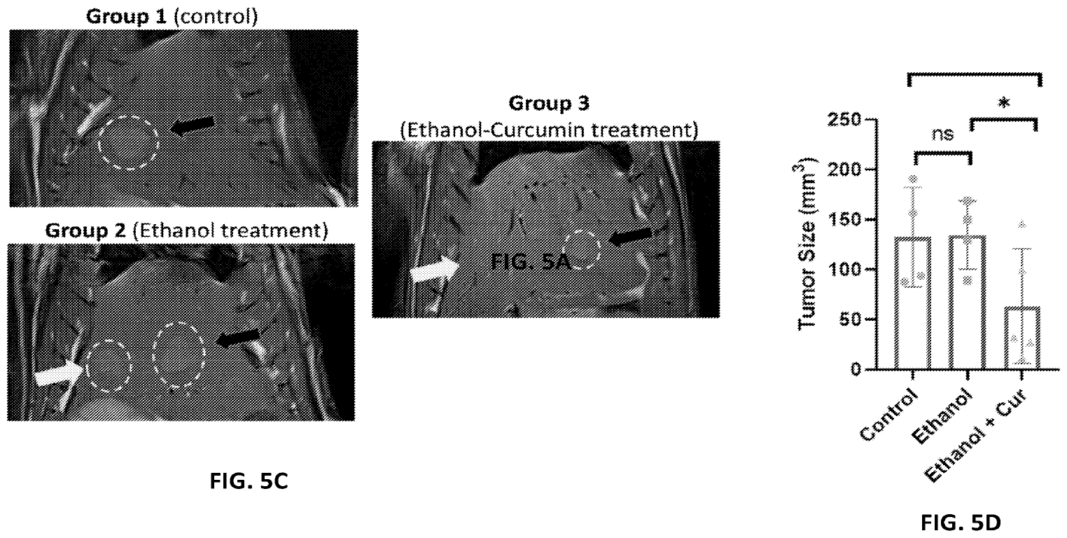

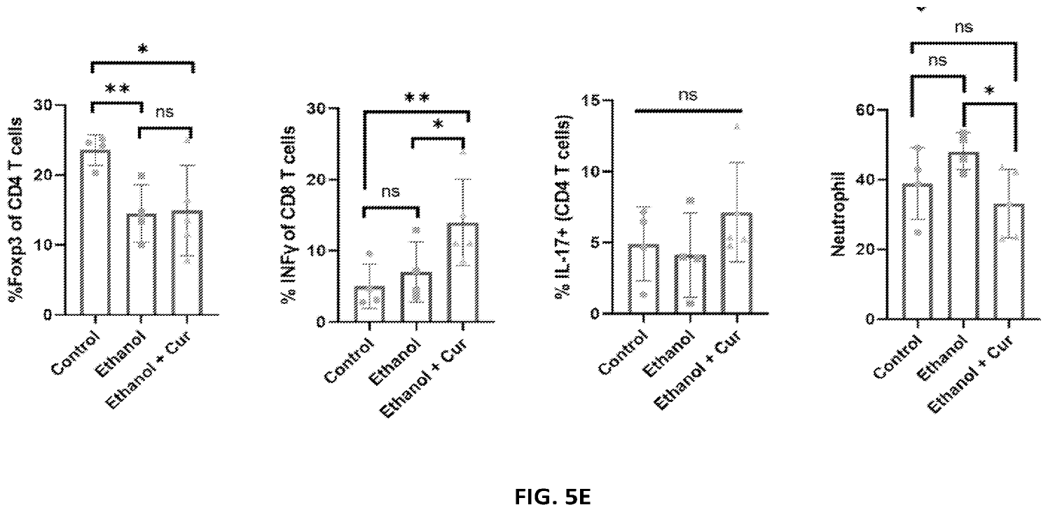

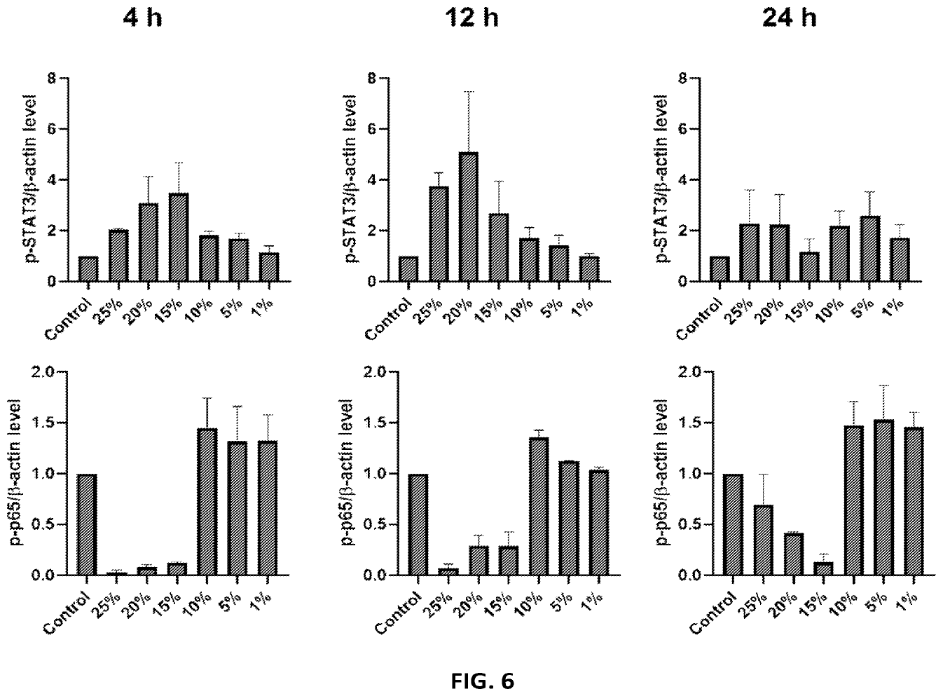

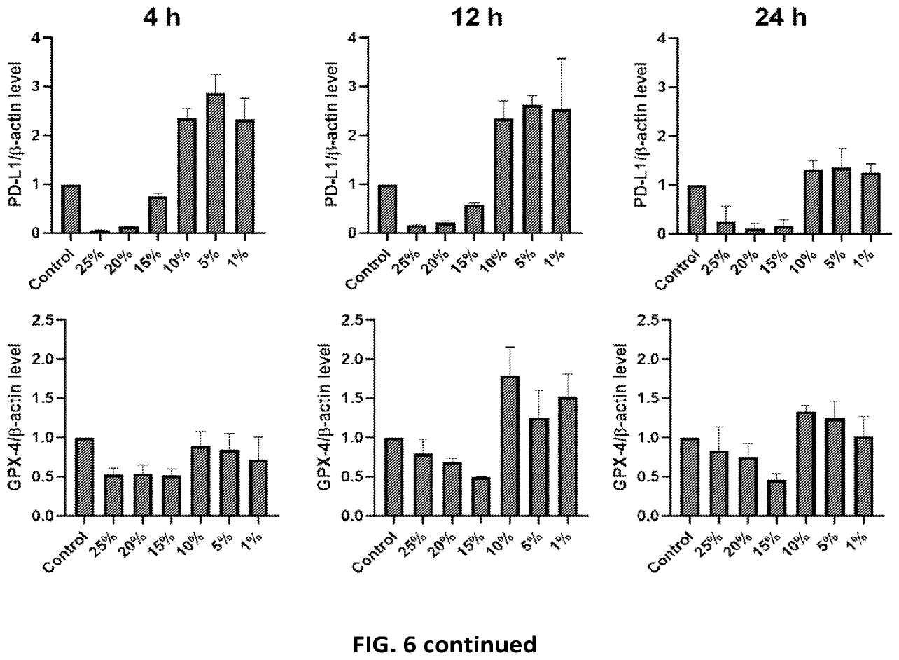

Enhanced cell death and iTME modulation of ethanol-curcumin solution as shown in the in vitro assays, was examined in vivo using a rat animal model. As shown in FIGS. 5A and 5B, simultaneously, the right lobe of rat liver was inoculated with non-treated N1S1 cells (5 million) only, and the left lobe was inoculated with a mixture of ethanol-curcumin treated N1S1 cells (2.5 million, 10% ethanol or 10% ethanol plus Cur2) and non-treated N1S1 cells (2.5 million). The rats in the control group were only inoculated in the right lobe of the liver with 5 million non-treated N1S1 cells. The right-side tumors (arrow in FIG. 5A) were established for testing abscopal effect. These tumors were collected for an immune status assay. The tumor in the control group was used as the baseline. MRI scanning showed tumors generated by orthotopically implanted N1S1 hepatoma cells in each group (FIG. 5C). The largest tumor volume was found in the control group with non-treated N1S1 cell implantation. Compared to the control (Group 1) and ethanol-treatment (Group 2), the tumor volume in the ethanol plus curcumin treatment group (Group 3) was the smallest. The average tumor volume of Group 3 was about 51 mm³ compared to about 130 mm³ of Group 1 and Group 2 (FIG. 5D). Further, as evidenced by a complete lack of tumor formation in the left liver lobe, the inoculation of ethanol plus curcumin-treated cells (Group 3) induced a greater control of local tumor growth than cells treated with ethanol alone (Group 2). This indicates that curcumin-ethanol ablation induced an effective abscopal effect compared to the ethanol ablation alone (Group 2). Finally, the immune status of the right-side tumor was investigated to analyze the potential anti-cancer immune effect of the integration of ethanol and curcumin on the microenvironment of distant tumors. As shown in FIG. 5E, treatment with both ethanol and ethanol plus curcumin led to the decreased population of Tregs as compared to the control group (Group 1). In addition, a significant increase of cytotoxic T lymphocytes (CTLs) was found in the ethanol and curcumin treatment group (Group 3). There was no significant difference among the three groups on IL-17 T cells. Although no significant difference was found between the control and ethanol treatment group on the neutrophil count, the combination ethanol-curcumin treatment significantly decreased the neutrophil population in the right-side tumor.

### Conclusion

Ethanol ablation, simply injecting ethanol into lesions, is one of the most reliable ablation modalities. It is commonly used and well-accepted for the treatment of small HCC. As demonstrated in other ablation procedures such as microwave ablation or cryoablation, ablation can induce necrosis and generate in situ tumoral antigens that may stimulate the immune system to inhibit potential recurrence during post-treatment immune surveillance. Current implementations of ethanol ablation therapy for HCC are significantly limited by incomplete tumor necrosis and tumor recurrence. In particular, post-ablation inflammatory tumor microenvironment (iTME) can hinder the therapeutic efficacy with adaptive mechanisms to evade immune cells. Modulating iTME is currently a promising strategy to improve anticancer therapeutic efficacy of the ethanol ablation therapy. In this study, the inventors demonstrated that ethanol treatment of various concentrations ranging from 25% to 5% inhibits liver cancer cell N1S1 growth in 10 min coincubation. However, these different ethanol concentrations induced incomplete cell death and iTME that contribute to the tumor progression. This information reflected one main drawback of current ethanol ablation procedure: heterogenous destruction of tumor cells, ranging from highest and most uniform cell death in the local injection site to lowest in the peripheral tumor region. Hence, enhanced anti-cancer effect and iTME modulation of the ethanol ablation is critically important to overcome this barrier and to improve therapeutic efficacy. Herein, the combination of ethanol and curcumin anti-cancer immune modulator achieved a synergistic anticancer effect as well as a modulation of p-STAT3, p-p65 and PD-L1 upregulation in N1S1 cells. Further, cellular uptake of curcumin was amplified by the ethanol solution and maintained in N1S1 hepatoma cells for at least 48 h. Increased anti-cancer effect and iTME modulation by ethanol-curcumin treatment were additionally observed in in vivo conditions by orthotopically implanting ethanol-curcumin treated N1S1 cells and non-treated secondary N1S1 cells in the rat liver. The abscopal effect of ethanol-curcumin treatment with the iTME modulation was demonstrated in the distant, non-treated N1S1-induced HCC. Overall, the demonstrated efficient combination of curcumin immune modulator and ethanol opens a new horizon to integrate the ethanol therapy procedure with the delivery of various immune modulators for enhancing anticancer immune response in the ethanol ablation procedure. The findings also show the potential of a simple ethanol ablation-based combination immunotherapy to modulate the iTME and effectively treat the primary and metastatic tumors using a local ablation therapy.

### REFERENCES

1. J. M. Llovet et al., Hepatocellular carcinoma. Nat Rev Dis Primers 7, 6 (2021).
2. N. J. Yi et al., Current role of surgery in treatment of early stage hepatocellular carcinoma: resection versus liver transplantation. Oncology 75 Suppl 1, 124-128 (2008).
3. C. Toso, G. Mentha, N. M. Kneteman, P. Majno, The place of downstaging for hepatocellular carcinoma. J Hepatol 52, 930-936 (2010).
4. A. Facciorusso, G. Serviddio, N. Muscatiello, Local ablative treatments for hepatocellular carcinoma: An updated review. World J Gastrointest Pharmacol Ther 7, 477-489 (2016).
5. R. Morhard et al., Development of enhanced ethanol ablation as an alternative to surgery in treatment of superficial solid tumors. Sci Rep 7, 8750 (2017).
6. T. S. Yamashita et al., Ultrasound-Guided Percutaneous Ethanol Ablation for Local Regional Recurrence of Medullary Thyroid Cancer. Am Surg 87, 1396-1399 (2021).
7. E. Armellini, S. F. Crino, M. Ballare, S. Pallio, P. Occhipinti, Endoscopic ultrasound-guided ethanol ablation of pancreatic neuroendocrine tumours: A case study and literature review. World J Gastrointest Endosc 8, 192-197 (2016).
8. J. Veselka et al., Long-term clinical outcome after alcohol septal ablation for obstructive hypertrophic cardiomyopathy: results from the Euro-ASA registry. Eur Heart J 37, 1517-1523 (2016).
9 E. L. Artifon et al., EUS-guided alcohol ablation of left adrenal metastasis from non-small-cell lung carcinoma. Gastrointest Endosc 66, 1201-1205 (2007).
10. M. Kuang et al., Ethanol ablation of hepatocellular carcinoma Up to 5.0 cm by using a multipronged injection needle with high-dose strategy. Radiology 253, 552-561 (2009).
11. M. Ryu et al., Therapeutic results of resection, transcatheter arterial embolization and percutaneous transhepatic ethanol injection in 3225 patients with hepatocellular carcinoma: a retrospective multicenter study. Jpn J Clin Oncol 27, 251-257 (1997).
12. R. Morhard et al., Development of enhanced ethanol ablation as an alternative to surgery in treatment of superficial solid tumors. Sci Rep-Uk 7, (2017).
13. M. Kuang et al., Ethanol Ablation of Hepatocellular Carcinoma Up to 5.0 cm by Using a Multipronged Injection Needle with High-Dose Strategy. Radiology 253, 552-561 (2009).
14. K. F. Chu, D. E. Dupuy, Thermal ablation of tumours: biological mechanisms and advances in therapy. Nat Rev Cancer 14, 199-208 (2014).
15. L. Shi et al., Inflammation induced by incomplete radiofrequency ablation accelerates tumor progression and hinders PD-1 immunotherapy. Nature Communications 10, 5421 (2019).
16. F. R. Greten, S. I. Grivennikov, Inflammation and Cancer: Triggers, Mechanisms, and Consequences. Immunity 51, 27-41 (2019).
17. C. Lu et al., Current perspectives on the immunosuppressive tumor microenvironment in hepatocellular carcinoma: challenges and opportunities. Mol Cancer 18, 130 (2019).
18. D.-H. Kim, J. Chen, R. A. Omary, A. C. Larson, MRI visible drug eluting magnetic microspheres for transcatheter intra-arterial delivery to liver tumors. Theranostics 5, 477-488 (2015).
19. W. Park et al., Development and Validation of Sorafenib-eluting Microspheres to Enhance Therapeutic Efficacy of Transcatheter Arterial Chemoembolization in a Rat Model of Hepatocellular Carcinoma. Radiology: Imaging Cancer 3, e200006 (2021).
20. K. N. Khan et al., Prospective analysis of risk factors for early intrahepatic recurrence of hepatocellular carcinoma following ethanol injection. Journal of Hepatology 32, 269-278 (2000).
21. A. Dey, A. I. Cederbaum, Alcohol and oxidative liver injury. Hepatology 43, S63-74 (2006).
22. J. P. de Koning et al., STAT3-mediated differentiation and survival and of myeloid cells in response to granulocyte colony-stimulating factor: role for the cyclin-dependent kinase inhibitor p27(Kipl). Oncogene 19, 3290-3298 (2000).
23. T. Yakovleva, I. Bazov, H. Watanabe, K. F. Hauser, G. Bakalkin, Transcriptional control of maladaptive and protective responses in alcoholics: a role of the NF-kappaB system. Brain Behav Immun 25 Suppl 1, S29-38 (2011).
24. K. Mors et al., Anti-inflammatory Effects of Alcohol Are Associated with JNK-STAT3 Downregulation in an In Vitro Inflammation Model in HepG2 Cells. Dis Markers 2021, 6622701 (2021).
25. L. Shi et al., Inflammation induced by incomplete radiofrequency ablation accelerates tumor progression and hinders PD-1 immunotherapy. Nat Commun 10, 5421 (2019).
26. J. Tan, T. Tang, W. Zhao, Z. S. Zhang, Y. D. Xiao, Initial Incomplete Thermal Ablation Is Associated With a High Risk of Tumor Progression in Patients With Hepatocellular Carcinoma. Front Oncol 11, 760173 (2021).
27. N. Zhang et al., Insufficient Radiofrequency Ablation Treated Hepatocellular Carcinoma Cells Promote Metastasis by Up-Regulation ITGB3. J Cancer 8, 3742-3754 (2017).
28. J. P. Erinjeri et al., Image-guided thermal ablation of tumors increases the plasma level of interleukin-6 and interleukin-10. Journal of vascular and interventional radiology 24, 1105-1112 (2013).
29. G. Mondal, S. Barui, S. Saha, A. Chaudhuri, Tumor growth inhibition through targeting liposomally bound curcumin to tumor vasculature. Journal of Controlled Release 172, 832-840 (2013).
30. Z. Xiao et al., Dual pH-sensitive nanodrug blocks PD-1 immune checkpoint and uses T cells to deliver NF-&#x3ba;B inhibitor for antitumor immunotherapy. Science Advances 6, eaay7785 (2020).

1. A composition for use in ethanol ablation of tumors, the composition comprising: ethanol and an anti-cancer immune modulator.

2. The composition of claim 1, wherein the anti-cancer immune modulator is curcumin.

3. The composition of claim 2, wherein the curcumin concentration is between about 0.1% w/v and about 5% w/v.

4. The composition of claim 2, wherein the curcumin concentration in the composition is between about 0.1% w/v and about 1% w/v.

5. The composition of any one of the proceeding claims, wherein the ethanol comprises between about 5% and about 30% of the composition.

6. The composition of any one of the preceding claims, wherein the ethanol comprises between about 5% and about 25% of the composition.

7. A method for treating a solid tumor in a subject in need thereof, the method comprising:
injecting into the tumor the composition of claim 1.

8. The method of claim 7, wherein the ethanol and the anti-cancer immune modulator are in an amount effective in combination to reduce the volume of the tumor.

9. The method of claim 7 or 8, wherein the treatment reduces the tumor volume by at least 50%.

10. The method of any one of claims 7-9, wherein the tumor is a cancerous tumor.

11. The method of any one of claims 7-10, wherein the tumor is a hepatocellular carcinoma (HCC) tumor, a thyroid tumor, a pancreatic tumor, a renal tumor, a prostate tumor, a lung tumor, a colorectal tumor, a colon tumor, or an adrenal tumor.

12. The method of claim 11, wherein the tumor is an HCC tumor.

13. The method of any one of claims 7-12, wherein the subject is human.

14. The method of any one of claims 7-13, wherein the ethanol and the anti-cancer immune modulator are in an amount effective in combination to prevent tumor formation or reduce the volume of tumors at other sites within the subject.

15. A method for increasing an immune response to a tumor in a subject in need thereof, the method comprising:
injecting into the tumor the composition of claim 1.

16. The method of claim 15, wherein the ethanol and the anti-cancer immune modulator are in an amount effective in combination to increase the immune response to the tumor.

17. The method of any one of claims 15-16, wherein the immune response is characterized by decreased activation of at least one of STAT3, p65, and PD-L1.

18. The method of any one of claims 15-17, wherein the tumor is a cancerous tumor.

19. The method of any one of claims 15-18, wherein the tumor is a hepatocellular carcinoma (HCC) tumor, a thyroid tumor, a pancreatic tumor, a renal tumor, a prostate tumor, a lung tumor, a colorectal tumor, a colon tumor, or an adrenal tumor.

20. The method of any one of claims 14-19, wherein the subject is human.

21. The method of any one of claims 7-20, wherein the anti-cancer immune modulator is curcumin.

22. The method of any one of claims 7-21, wherein the curcumin concentration is between about 0.1% w/v and about 5% w/v.

23. The method of any one of claims 7-22, wherein the curcumin concentration in the composition is between about 0.1% w/v and about 1% w/v. 24 The method of any one of claims 7-23, wherein the ethanol comprises between about 5% and about 30% of the composition.

25. The method of any one of claims 7-24, wherein the ethanol comprises between about 5% and about 25% of the composition.
